(19) United States
(12) Patent Application Publication
(10) Pub. No.: US 2022/0354575 A1
(43) Pub. Date: Nov. 10, 2022

(54) LASER PULSE SELECTION USING MOTORIZED SHUTTER

(71) Applicant: Alcon Inc., Fribourg (CH)

(72) Inventors: David Jung, Tustin, CA (US); Daniel Castro, Lake Forest, CA (US); Zsolt Bor, San Clemente, CA (US); Corey Stewart, Irvine, CA (US); Billy Lee, Lake Forest, CA (US)

(21) Appl. No.: 17/662,153

(22) Filed: May 5, 2022

Related U.S. Application Data
(60) Provisional application No. 63/186,387, filed on May 10, 2021.

(51) Int. Cl. A61B 18/20; G03F 7/20; G02F 1/13363

(52) U.S. Cl. CPC A61B 18/203; G03F 7/70041; G02F 1/133638; A61B 2018/20359; A61B 2018/20554

(57) ABSTRACT

Systems and methods are disclosed for selectively allowing or preventing output of laser pulses. In some embodiments, a laser system comprises a shutter and a shutter motor. The shutter motor is configured to move the shutter in an alternating manner between a first position in which output of laser electromagnetic radiation is allowed a second position in which output of laser electromagnetic radiation is prevented.

### TECHNICAL FIELD

The present disclosure is directed to systems and methods for selectively allowing or preventing output of laser pulses.

### BACKGROUND

Lasers are used in many different medical procedures including a number of different ophthalmic procedures. For example, lasers may be used in cataract surgery, such as for fragmenting the cataractous lens. In some procedures, a laser is used for initial fragmentation of the lens, followed by phacoemulsification of the lens by an ultrasonic handpiece to complete the breakdown of the lens for removal. In other procedures, the laser may be used for complete fragmentation or phacoemulsification of the lens for removal, without the need for a separate application of ultrasonic energy. Lasers may also be used for other steps in cataract surgery, such as for making the corneal incision(s) and/or opening the capsule.

Lasers may also be used in vitreoretinal surgery. In some procedures, a laser may be used for vitrectomy, to sever or break the vitreous fibers for removal. The laser may be incorporated into a vitrectomy probe, and the energy from the laser may be applied to the vitreous fibers to sever or break the vitreous fibers for removal.

In other vitreoretinal applications, lasers may be used for photocoagulation of retinal tissue. Laser photocoagulation may be used to treat issues such as retinal tears and/or the effects of diabetic retinopathy.

U.S. Patent Application Publication No. 2018/0360657 discloses examples of an ophthalmic laser system. That application describes laser uses such as for forming surgical cuts or for photodisrupting ophthalmic tissue as well as for cataract surgery, such as laser-assisted cataract surgery (LACS). U.S. Patent Application Publication No. 2019/0201238 discloses other examples of an ophthalmic laser system. That application describes laser uses such as in a vitrectomy probe for severing or breaking vitreous fibers. U.S. Patent Application Publication No. 2018/0360657 and U.S. Patent Application Publication No. 2019/0201238 are expressly incorporated by reference herein in their entirety.

Some laser systems emit pulses, with the pulses having a desired duration and repetition rate. Operating a laser in pulses can achieve desirable power and energy characteristics for a particular application. In addition, while the energy of a beam emitted by a laser can be controlled by controlling the laser itself, in some systems it is desirable to control the amount of energy of a laser beam downstream from the laser. Existing systems for laser pulse selection typically have one or more drawbacks, such as power loss, complexity, cost, etc. There is a need for improved systems and methods for laser pulse selection.

### SUMMARY

The present disclosure is directed to improved systems and methods for selectively allowing or preventing output of laser electromagnetic energy.

In some embodiments, a laser system comprises a laser configured to emit electromagnetic radiation and a laser shutter assembly comprising a shutter and a shutter motor. The shutter motor is configured to move the shutter in an alternating manner between a first position in which electromagnetic radiation emitted by the laser is allowed to be output from the laser system and a second position in which electromagnetic radiation emitted by the laser is prevented from being output from the laser system. The laser may be configured to emit electromagnetic radiation in pulses.

In some embodiments, in the first position the shutter is positioned out of a path of electromagnetic radiation emitted by the laser and in the second position the shutter is positioned in the path of the electromagnetic radiation emitted by the laser.

In some embodiments, in the first position the shutter is positioned in a path of the electromagnetic radiation emitted by the laser and in the second position the shutter is positioned out of the path of electromagnetic radiation emitted by the laser.

In some embodiments, in the first position the shutter is positioned in a first orientation in a path of the electromagnetic radiation emitted by the laser and in the second position the shutter is positioned in a second orientation in the path of electromagnetic radiation emitted by the laser, wherein the second orientation is different from the first orientation.

In some embodiments, the laser system further comprises a controller adapted to send signals to a shutter motor driver to control moving the shutter between the first position and the second position.

In some embodiments, the shutter may comprise a mirror, and the shutter motor may comprise a galvanometer motor. The galvanometer motor may be configured to move the mirror between the first position and the second position by rotating the mirror about a mirror axis by a selected angle. The mirror axis and the path of the electromagnetic radiation adjacent the mirror may be in a skew line relationship with respect to each other.

In some embodiments, the laser system may further comprise a laser energy control system configured to regulate the amount of electromagnetic energy of each laser pulse that exits the laser system. The laser energy control system may comprise a waveplate, a waveplate motor, and a polarizer plate, wherein the waveplate motor is configured to move the waveplate into different positions corresponding to different percentages of laser electromagnetic energy permitted to pass through the laser energy control system.

In some embodiments, a method of controlling a laser system comprises emitting electromagnetic radiation from a laser in a path and moving a shutter in an alternating manner between a first position in which electromagnetic radiation emitted by the laser is output from the laser system and a second position in which electromagnetic radiation emitted by the laser is not output from the laser system. The electromagnetic radiation may be emitted from the laser in pulses.

In some embodiments, the method may further comprise sending signals to a shutter motor driver from a controller to control moving the shutter between the first position and the second position.

In some embodiments, moving the shutter in an alternating manner between the first position and the second position may comprise a galvanometer motor rotating a mirror about a mirror axis back and forth between the first position to the second position.

In some embodiments, the method may further comprise moving a waveplate in the path of the electromagnetic radiation emitted by the laser into different positions to regulate the amount of electromagnetic energy of each laser pulse that exits the laser system. The different positions of the waveplate may correspond to different percentages of laser electromagnetic energy permitted to be output from the laser system.

Further examples and features of embodiments of the invention will be evident from the drawings and detailed description.

### BRIEF DESCRIPTION OF THE DRAWINGS

The accompanying drawings illustrate example implementations of the devices and methods disclosed herein and, together with the description, serve to explain the principles of the present disclosure.

FIG. 1 shows a schematic view of an example of a laser system in accordance with the disclosure, with a shutter of the laser system in a first position in which electromagnetic radiation emitted by a laser is allowed to be output from the laser system.

FIG. 2 shows a schematic view of the example laser system of FIG. 1, with the shutter of the laser system in a second position in which electromagnetic radiation emitted by the laser is prevented from being to be output from the laser system.

FIG. 3 shows an example of a shutter and shutter motor with the shutter in a first orientation.

FIG. 4 shows the shutter and shutter motor of FIG. 3 with the shutter in a second orientation.

FIG. 5 shows a schematic view of another example of a laser system in accordance with the disclosure, with a shutter of the laser system in a first position in which electromagnetic radiation emitted by a laser is allowed to be output from the laser system.

FIG. 6 shows a schematic view of the example laser system of FIG. 5, with the shutter of the laser system in a second position in which electromagnetic radiation emitted by the laser is prevented from being to be output from the laser system.

FIG. 7 shows a schematic view of another example of a laser system in accordance with the disclosure, with a shutter of the laser system in a first position in which electromagnetic radiation emitted by a laser is allowed to be output from the laser system.

FIG. 8 shows a schematic view of the example laser system of FIG. 7, with the shutter of the laser system in a second position in which electromagnetic radiation emitted by the laser is prevented from being to be output from the laser system.

FIG. 9 shows a schematic view of another example of a laser system in accordance with the disclosure, with a shutter of the laser system in a first position in which electromagnetic radiation emitted by a laser is allowed to be output from the laser system.

FIG. 10 shows a schematic view of the example laser system of FIG. 9, with the shutter of the laser system in a second position in which electromagnetic radiation emitted by the laser is prevented from being to be output from the laser system.

FIG. 11 illustrates an example shutter control process.

The accompanying drawings may be better understood by reference to the following detailed description.

### DETAILED DESCRIPTION

For the purposes of promoting an understanding of the principles of the disclosure, reference will now be made to the implementations illustrated in the drawings, and specific language will be used to describe those implementations and other implementations. It will nevertheless be understood that no limitation of the scope of the claims is intended by the examples shown in the drawings or described herein. Any alterations and further modifications to the illustrated or described systems, devices, instruments, or methods, and any further application of the principles of the present disclosure, are fully contemplated as would normally occur to one skilled in the art to which the disclosure relates. In particular, the features, components, and/or steps described with respect to one implementation of the disclosure may be combined with features, components, and/or steps described with respect to other implementations of the disclosure. For simplicity, in some instances the same reference numbers are used throughout the drawings to refer to the same or like parts.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

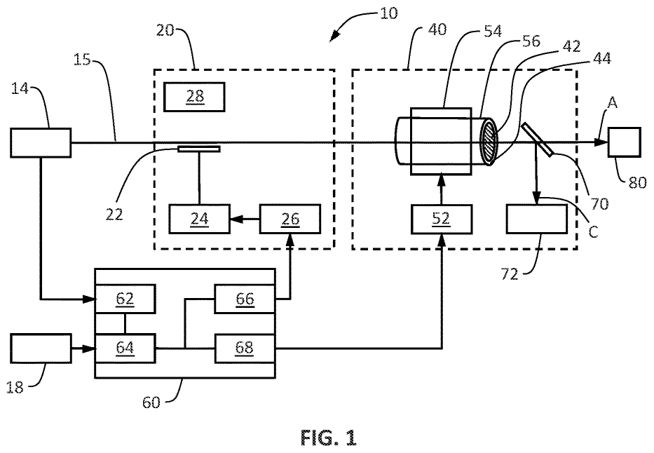

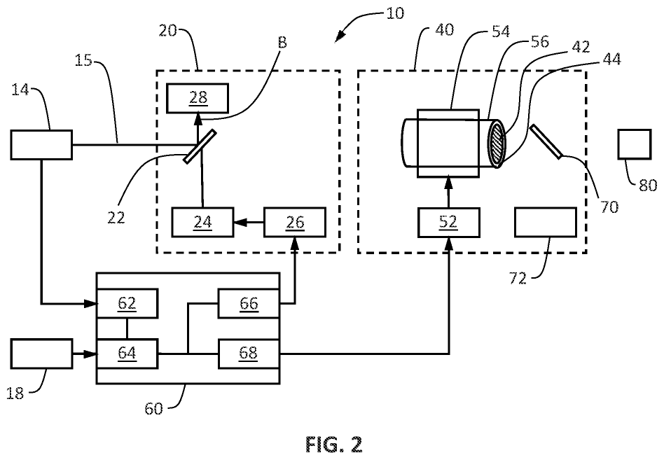

FIGS. 1 and 2 show schematic views of an example laser system 10 in accordance with the disclosure. FIG. 1 shows the laser system 10 with a shutter 22 of the laser system 10 in a first position in which electromagnetic radiation emitted by a laser 14 is allowed to be output from the laser system 10. In this embodiment, in the first position the shutter 22 is positioned out of a path 15 of electromagnetic radiation emitted by a laser 14. FIG. 2 shows the laser system 10 with the shutter 22 of the laser system 10 in a second position in which electromagnetic radiation emitted by the laser 14 is prevented from being output from the laser system 10. In this embodiment, in the second position the shutter 22 is positioned in the path 15 of the electromagnetic radiation emitted by the laser 14.

As shown in FIGS. 1 and 2, the example laser system 10 comprises a laser 14, a laser shutter assembly 20, and an optional laser energy control system 40. If desired and depending upon the application, the laser system 10 may also comprise one or more other optical components or other components. The laser 14 is configured to emit electromagnetic radiation in pulses. In operation, the laser 14 emits laser electromagnetic radiation in pulses along a laser path 15. When permitted by the shutter 22 as described below, the laser electromagnetic energy exits an output of the system 10 and is directed to a target 80. The target 80 may be another optical component, such as an optical fiber, lens, or other component, or the target 80 may be an ultimate target of laser energy. For example, the target 80 may be ophthalmic tissue, such as a cataractous lens, vitreous fibers, retinal tissue, or other tissue.

In the illustrated example, the laser shutter assembly 20 comprises a shutter 22, a shutter motor 24, and a shutter motor driver 26. The shutter motor 22 is configured to move the shutter 22 in an alternating manner between the position shown in FIG. 1, in which the shutter 22 is positioned out of the path 15 of the electromagnetic radiation emitted by the laser 14, and the position shown in FIG. 2, in which the shutter 22 is positioned in the path 15 of the electromagnetic radiation emitted by the laser 14.

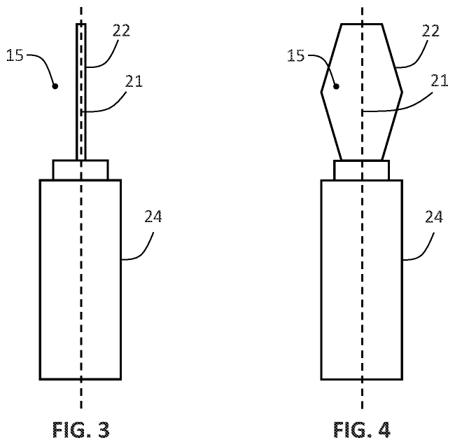

An example shutter motor 24 and shutter 22 assembly is illustrated in FIGS. 3 and 4. The shutter motor 24 may be any suitable motor capable of moving the shutter 22 in the desired manner, and the shutter 22 may be any suitable shutter adapted to block or redirect electromagnetic radiation from the laser 14 when the shutter 22 is positioned in the path 15 of the electromagnetic radiation emitted by the laser 14.

In one example, the shutter motor 24 and shutter 22 may be a galvo mirror comprising a galvanometer motor as the shutter motor 24 and a mirror as the shutter 22. Example galvo mirrors that may be used in a laser system such as laser system 10 include galvo mirrors supplied by ScannerMAX, a division of Pangolin Laser Systems, Inc., such as the Compact-506 Galvo, as well as others.

The shutter motor 24 (e.g., galvanometer motor) is capable of rapidly moving the shutter 22 (e.g., mirror) back and forth between the first position and the second position. In the example of FIGS. 3 and 4, the shutter 22 is rotated over a selected angle of rotation by the shutter motor 24 about a shutter axis 21. The shutter axis 21 in this example is offset from the laser path 15. In FIGS. 3 and 4, the laser path 15 is perpendicular to the plane of the drawing, heading into the drawing page. In the example of FIGS. 3 and 4, the shutter (mirror) axis 21 and the path 15 of the electromagnetic radiation adjacent the mirror are in a skew line relationship with respect to each other (i.e., they are lines in different planes). The shutter motor 24 (e.g., galvanometer motor) is capable of moving the shutter 22 (e.g., mirror) by a selected angle of rotation to move the mirror between the first position, shown in FIG. 3, in which in the embodiment of FIGS. 1-2 the shutter 22 is out of the path 15 of the laser energy, and the second position, shown in FIG. 4, in which in the embodiment of FIGS. 1-2 the shutter 22 is in the path 15 of the laser energy.

As can be seen in FIG. 1, in the laser system 10, when the shutter 22 is in the first position, the shutter 22 is out of the path 15 of the laser electromagnetic radiation, thereby not obstructing or redirecting it. When the shutter 22 is in the first position, the laser electromagnetic radiation is permitted to be output from the laser system 10 to continue toward the target 80, in the direction indicated by arrow A. The direction indicated by arrow A designates the direction from the laser 14 to the target 80 and may be, but is not required to be, a straight line. For example, in some embodiments, one or more optical components may redirect the laser energy between the laser 14 and the target 80 such that the direction indicated by arrow A is not a straight line.

As can be seen in FIG. 2, in the laser system 10, when the shutter 22 is in the second position, the shutter 22 is in the path 15 of the laser electromagnetic radiation, thereby obstructing or redirecting it. The shutter 22 may absorb and/or reflect the laser electromagnetic radiation. In this embodiment, when the shutter 22 is in the second position, the shutter 22 reflects the laser electromagnetic radiation in the direction of arrow B to a beam dump 28 designed to absorb and/or diffuse it. The direction indicated by arrow B designates the direction from the laser 14 to the beam dump 28, and, like the direction indicated by arrow A, may or may not be a straight line, as one or more optical components may redirect the laser energy between the laser 14 and the beam dump 28. For example, in this embodiment, the shutter 22, when in the second position, redirects the laser energy to the beam dump 28. Various beam dumps are known and available, having features for absorbing and/or diffusing laser electromagnetic energy, e.g., matte black color, ridges, metal, or other characteristics. In some embodiments, the shutter 22 may be designed to absorb and/or diffuse laser electromagnetic energy, with or without a beam dump 28. As can be seen in FIG. 2, when the shutter 22 is in the second position, the laser electromagnetic radiation is prevented from being output from the laser system 10.

As shown in FIGS. 1 and 2, the example laser system 10 may comprise a laser energy control system 40. The laser energy control system 40 comprises a waveplate 42 and a mechanism for moving the waveplate 42. The mechanism for moving the waveplate may comprise a waveplate motor 54 which includes a hollow motor shaft 56. The waveplate 42 is mounted on one end of the hollow motor shaft 56, and a waveplate adaptor 44 may be used to mount the waveplate 42 to the hollow motor shaft 56. The components of the laser energy control system 40 are arranged such that laser electromagnetic radiation from the laser 14 enters the hollow motor shaft 56 at one end, passes through the hollow motor shaft 56, and then exits through the waveplate 42 at the other end of the hollow motor shaft 56. The laser energy control system 40 further comprises a waveplate motor driver 52 for the waveplate motor 54. In operation, the waveplate motor 54 causes rotation of the hollow motor shaft 56 in a desired amount of angular movement which thereby causes rotation of the waveplate 42 in the desired amount of angular movement.

The waveplate 42 works with a polarizer plate 70 to pass or block laser energy in an amount controlled by the rotation of the waveplate 42. The laser energy, which is polarized, passes through the waveplate 42, which in turn rotates the polarized laser beam anywhere from 0 to 90 degrees, based on the rotational position of the waveplate 42. After passing through the waveplate 42, the laser energy reaches the polarizer plate 70, which allows to pass laser energy that is polarized in one polarization plane and reflects any laser energy that has other polarizations. Laser energy that is reflected by the polarizer plate 70 may be directed in the direction C to a beam dump 72. The direction indicated by arrow C designates the direction from the polarizer plate 70 to the beam dump 72, and, like the directions indicated by arrows A and B, may or may not be a straight line, as one or more optical components may redirect the laser energy between the polarizer plate 70 and the beam dump 72.

The operating positions of the waveplate 42 may be incremental positions along a 90 degree arc. All the way to one side of the arc, the waveplate 42 may change the polarity to be rotated 90 degrees with respect to the polarization permitted by the polarizer plate 70 (or, in an embodiment in which the laser electromagnetic radiation enters the waveplate 42 already rotated 90 degrees with respect to the polarization permitted by the polarizer plate 70, the waveplate 42 may leave the polarity unchanged). When laser energy that has its polarity oriented 90 degrees with respect to the polarization permitted by the polarizer plate 70 strikes the polarizer plate 70, the polarizer plate 70 reflects that laser energy. All the way to the other side of the arc, the waveplate 42 may change the polarity to be in the same plane as the polarization permitted by the polarizer plate 70 (or, in an embodiment in which the laser electromagnetic radiation enters the waveplate 42 already in the same plane as the polarization permitted by the polarizer plate 70, the waveplate 42 may leave the polarity unchanged). When laser energy that has its polarity oriented in the same plane as the polarization permitted by the polarizer plate 70 strikes the polarizer plate 70, the polarizer plate 70 allows that laser energy to pass through. In intermediate angular positions along the 90 degree arc, the waveplate 42 changes the polarity of the laser electromagnetic radiation in increments between being oriented in the same plane as the polarization permitted by the polarizer plate 70 and being rotated 90 degrees with respect to that plane. Thus, the waveplate 42 in combination with the polarizer plate 70 allows anywhere from 0% to 100% of the laser energy to pass through to the output of the laser system 10 to the target 80, depending upon the angular position of the waveplate 42.

The example laser system 10 shown in FIGS. 1 and 2 also comprises a controller 60 for controlling operation of the shutter 22 and, if implemented, the waveplate 42. In the illustrated embodiment, the controller 60 comprises a trigger input 62, a control data processor 64, a shutter motor control 66, and a waveplate control 68.

The trigger input 62 receives signals regarding the timing of the laser pulses. The control data processor 64 receives input from a system control 18 regarding the desired output for the laser system 10, which is used to control the shutter 22 and, if implemented, the waveplate 42. The control data processor 64 also receives signals from the trigger input 62 indicating the timing of the laser pulses.

The system control 18 provides input to the control data processor 64 based upon the desired mode of operation, which may be selected through user control or through automatic control. For example, the mode of operation may be for a certain level of laser energy output, which can be controlled by allowing all of the laser pulses to pass to the system output, none of the laser pulses to pass to the system output, or a certain percentage of laser pulses to pass to the system output. For example, the desired level of laser energy output may correspond to allowing one pulse out of every ten pulses to pass through, two pulses out of every ten pulses to pass through, three pulses out of every ten pulses to pass through, and so on. Stated another way, the desired level of laser energy output may correspond to allowing 10%, 20%, 30%, and so on, of the laser pulses to pass through. The desired level of laser energy output may also correspond to allowing different sequences of laser pulses to pass through. For example, the desired level of laser energy output may correspond to a sequence of allowing one laser pulse, then disallowing one laser pulse, then allowing two laser pulses, then disallowing one laser pulse, and then repeating this sequence. Many other examples and variations are possible.

Based on the input from the system control 18 regarding the desired output for the laser system 10 and the signals from the trigger input 62, the control data processor 64 sends signals to the shutter motor control 66 to in turn send signals to the shutter motor driver 26 to control the movement of the shutter motor 24 and shutter 22. By controlling the shutter 22, the controller 60 controls whether or not a laser pulse emitted by the laser goes to the output of the laser system. This control may be on a pulse-by-pulse basis, or for groups of pulses at a time. In the illustrated example, the control data processor 64 also sends signals to the waveplate control 68 to in turn send signals to the waveplate motor driver 52 to control the movement of the waveplate motor 54 and waveplate 42. By controlling the waveplate 42, the controller 60 controls the amount of energy of each laser pulse that goes to the output of the laser system.

In addition to controller 60, a laser system as disclosed herein may include other computer and electrical components as known in the art for controlling the system. The computer components may include one or more processors, memory components, and hardware and/or software components.

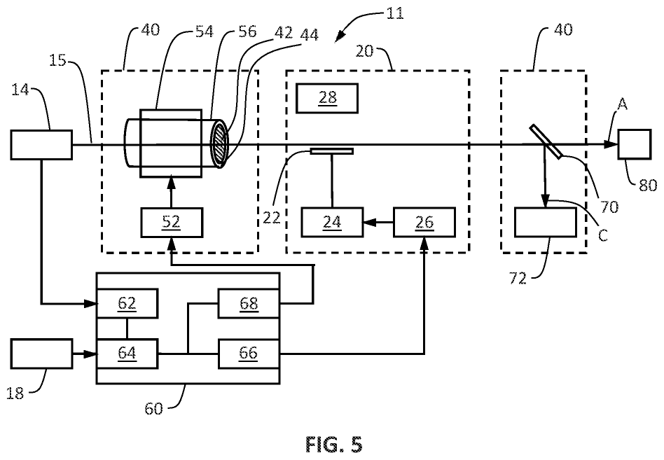

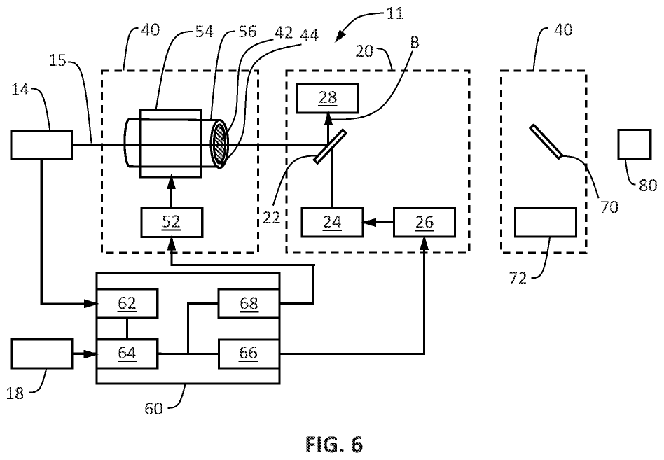

FIGS. 5 and 6 show schematic views of another example laser system 11 in accordance with the disclosure. Components in FIGS. 5 and 6 that are the same as in FIGS. 1 and 2 are designated with the same reference numbers. FIG. 5 shows the laser system 11 with a shutter 22 of the laser system 11 in a first position in which electromagnetic radiation emitted by a laser 14 is permitted to be output from the laser system 11. FIG. 6 shows the laser system 11 with the shutter 22 of the laser system 11 in a second position in which electromagnetic radiation emitted by the laser 14 is prevented from being output from the laser system 11.

The laser system 11 in FIGS. 5 and 6 is similar to the laser system 10 in FIGS. 1 and 2 except that in laser system 11 part of the laser energy control system 40 is positioned after the laser 14 and before the laser shutter assembly 20. In this example, the waveplate 42, the waveplate motor 54 with the hollow motor shaft 56, and the waveplate adaptor 44 are positioned after the laser 14 and before the laser shutter assembly 20. In the illustrated example, the polarizer plate 70 is located after the laser shutter assembly 20, but the polarizer plate 70 may alternatively be located before the laser shutter assembly 20. The components of the laser system 11 operate similarly as described above with respect to laser system 10.

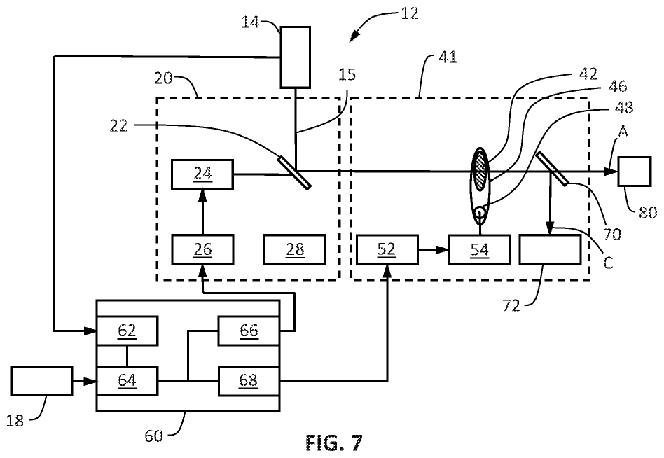

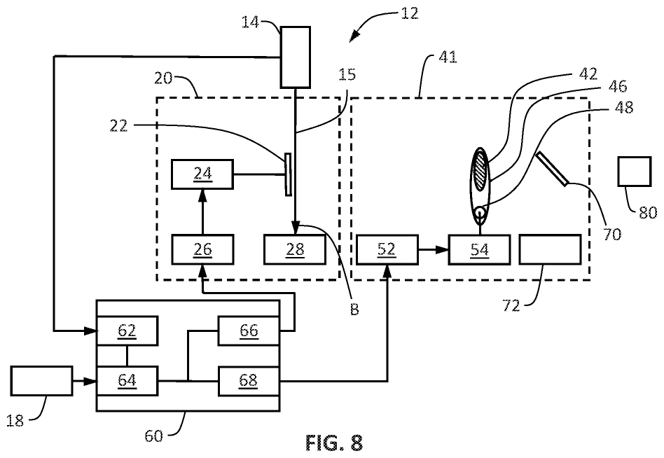

FIGS. 7 and 8 show schematic views of another example laser system 12 in accordance with the disclosure. Components in FIGS. 7 and 8 that are the same as in FIGS. 1 and 2 are designated with the same reference numbers. FIG. 7 shows the laser system 12 with a shutter 22 of the laser system 12 in a first position in which electromagnetic radiation emitted by a laser 14 is permitted to be output from the laser system 12. FIG. 8 shows the laser system 12 with the shutter 22 of the laser system 12 in a second position in which electromagnetic radiation emitted by the laser 14 is prevented from being output from the laser system 12.

The laser system 12 in FIGS. 7 and 8 is similar to the laser system 10 in FIGS. 1 and 2 except that the shutter in laser system 12 is arranged such that when the shutter is in the first position, shown in FIG. 7, the shutter 22 is positioned in a path 15 of the electromagnetic radiation emitted by the laser 14, and the shutter reflects the laser energy toward the output of the laser system 12 and the target 80 in the direction indicated at arrow A, and such that when the shutter 22 is in the second position, shown in FIG. 8, the shutter 22 is positioned out of the path 15 of electromagnetic radiation emitted by the laser 14, and the laser energy proceeds to the beam dump 28 in the direction indicated at arrow B.

In other respects, the laser system 12 is similar to the laser system 10. The laser system 12 comprises a laser 14, a laser shutter assembly 20, and an optional laser energy control system 41. The laser system 12 may also comprise one or more other optical components or other components. In operation, the laser 14 emits laser electromagnetic radiation in pulses along a laser path 15, whereby, when the shutter 22 is in the first position, the laser energy exits an output of the laser system 12 and is directed to a target 80, and when the shutter 22 is in the second position, the laser energy is not output from the laser system 12.

As in laser system 10, the laser shutter assembly 20 in laser system 12 comprises a shutter 22, a shutter motor 24, and a shutter motor driver 26. The shutter motor 22 is configured to move the shutter 22 in an alternating manner between the first position, shown in FIG. 7, and the second position, shown in FIG. 8. The laser system 12 may use a shutter motor 24 and shutter 22 similar to those described above with respect to laser system 10, including the shutter motor 24 and shutter 22 assembly illustrated in FIGS. 3 and 4.

The laser system 12 may have a laser energy control system similar to the laser energy control system 40 described above with respect to laser system 10. An alternative laser energy control system 41, which may be used in other laser system embodiments described herein, is illustrated in FIGS. 7 and 8.

The laser energy control system 41 comprises a waveplate 42 and a mechanism for moving the waveplate 42. The mechanism for moving the waveplate 42 may comprise a waveplate motor 54, a gear or pulley 48, and a belt 46. The laser energy control system 41 further comprises a waveplate motor driver 52 for the waveplate motor 54. The belt 46 extends around the gear or pulley 48 and the waveplate 42 (or a carriage carrying the waveplate 42), such that rotation of the gear or pulley 48 by the waveplate motor 54 drives rotation of the waveplate 42. The waveplate motor 54 may be a stepper motor, although other suitable motors such as voice-coil and other motors may be used. In operation, the waveplate motor 54 drives the gear or pulley 48 which in turn drives the belt 46 and thereby causes rotation of the waveplate 42 in the desired amount of angular movement.

The waveplate 42 works with a polarizer plate 70 to pass or block laser energy in an amount controlled by the rotation of the waveplate 42, in a similar manner as described above with respect to laser energy control system 40. The waveplate 42 in combination with the polarizer plate 70 allows anywhere from 0% to 100% of the laser energy to pass through to the output of the laser system 12 to the target 80, depending upon the angular position of the waveplate 42. Laser energy that is reflected by the polarizer plate 70 may be directed in the direction C to a beam dump 72, as shown in FIG. 7.

The laser system 12 may have a controller 60 similar to the controller described above with respect to laser system 10. The controller 60 comprises a trigger input 62, a control data processor 64, a shutter motor control 66, and a waveplate control 68, all operating in a similar manner as described above. One difference in laser system 12 as compared to laser system 10 is that when a laser pulse is to be prevented from proceeding to the output of the laser system 12, the controller 60 via shutter motor control 66 sends a signal to put the shutter out of the laser path, as shown in FIG. 8, and when a laser pulse is to be permitted to proceed to the output of the laser system 12, the controller 60 via shutter motor control 66 sends a signal to put the shutter in the laser path, as shown in FIG. 7.

In alternative embodiments, similar as described above with respect to FIGS. 5 and 6, all or part of the laser energy control system 41 in laser system 12 may be positioned after the laser 14 and before the laser shutter assembly 20. For example, the waveplate 42, the waveplate motor 54, the gear or pulley 48, and the belt 46 may be positioned after the laser 14 and before the laser shutter assembly 20. In addition, the polarizer plate 70 may be located before or after the laser shutter assembly 20.

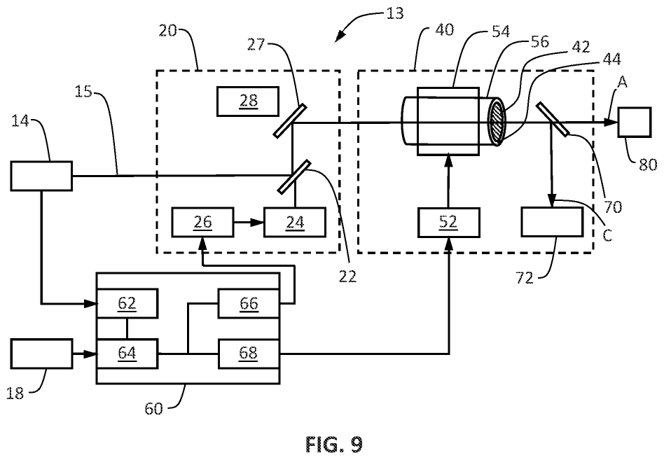

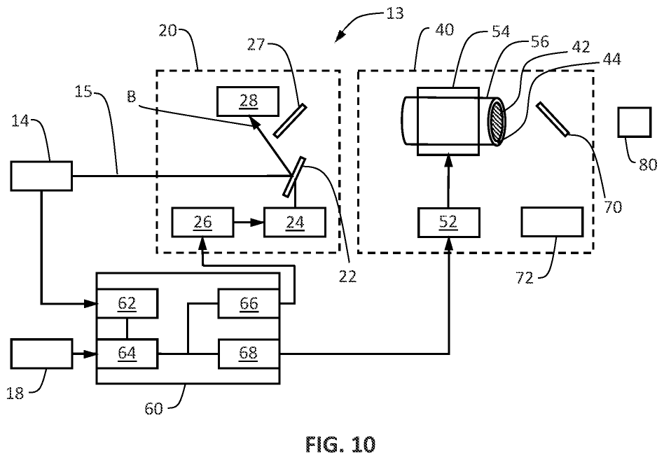

FIGS. 9 and 10 show schematic views of another example laser system 13 in accordance with the disclosure. Components in FIGS. 9 and 10 that are the same as in FIGS. 1 and 2 are designated with the same reference numbers. FIG. 9 shows the laser system 13 with a shutter 22 of the laser system 13 in a first position in which electromagnetic radiation emitted by a laser 14 is permitted to be output from the laser system 13, reflected by the shutter 22 and an optional mirror 27. FIG. 10 shows the laser system 13 with the shutter 22 of the laser system 13 in a second position in which electromagnetic radiation emitted by the laser 14 is prevented from being output from the laser system 13, reflected by the shutter 22 to a beam dump 28.

The laser system 13 in FIGS. 9 and 10 is similar to the laser system 10 in FIGS. 1 and 2 except that the shutter in laser system 13 is arranged such that when the shutter is in the first position, shown in FIG. 9, the shutter 22 is positioned in a path 15 of the electromagnetic radiation emitted by the laser 14, and the shutter reflects the laser energy toward the output of the laser system 11 and the target 80 in the direction indicated at arrow A, and such that when the shutter 22 is in the second position, shown in FIG. 10, the shutter 22 is also positioned in the path 15 of electromagnetic radiation emitted by the laser 14 but in a different orientation, such that the shutter 22 reflects the laser energy to the beam dump 28 in the direction indicated at arrow B.

In other respects, the laser system 13 is similar to the laser system 10. The laser system 13 comprises a laser 14, a laser shutter assembly 20, and an optional laser energy control system 40. The laser system 13 may also comprise one or more other optical components or other components. In operation, the laser 14 emits laser electromagnetic radiation in pulses along a laser path 15, whereby, when the shutter 22 is in the first position, the laser energy exits an output of the laser system 13 and is directed to a target 80, and when the shutter 22 is in the second position, the laser energy is not output from the laser system 13.

As in laser system 10, the laser shutter assembly 20 in laser system 13 comprises a shutter 22, a shutter motor 24, and a shutter motor driver 26. The shutter motor 22 is configured to move the shutter 22 in an alternating manner between the first position, shown in FIG. 9, and the second position, shown in FIG. 10. The laser system 13 may use a shutter motor 24 and shutter 22 similar to those described above with respect to laser system 10, including the shutter motor 24 and shutter 22 assembly illustrated in FIGS. 3 and 4.

The laser system 13 may have a laser energy control system similar to the laser energy control system 40 described above with respect to laser system 10. Like the laser systems 10 and 11, the laser system 13 alternatively may use a laser energy control system like the laser energy control system 41 illustrated in FIGS. 7 and 8.

The laser system 13 may have a controller 60 similar to the controller described above with respect to laser system 10. The controller 60 comprises a trigger input 62, a control data processor 64, a shutter motor control 66, and a waveplate control 68, all operating in a similar manner as described above. One difference in laser system 13 as compared to laser system 10 is that when a laser pulse is to be permitted to proceed to the output of the laser system 13, the controller 60 via shutter motor control 66 sends a signal to put the shutter 22 in the laser path in a first orientation as shown in FIG. 9, and when a laser pulse is to be prevented from proceeding to the output of the laser system 13, the controller 60 via shutter motor control 66 sends a signal to put the shutter 22 in the laser path in a second orientation, as shown in FIG. 10, wherein the second orientation is different from the first orientation.

In alternative embodiments, similar as described above with respect to FIGS. 5 and 6, all or part of the laser energy control system 40 in laser system 13 may be positioned after the laser 14 and before the laser shutter assembly 20. For example, the waveplate 42, the waveplate motor 54 with the hollow motor shaft 56, and the waveplate adaptor 44 may be positioned after the laser 14 and before the laser shutter assembly 20. In addition, the polarizer plate 70 may be located before or after the laser shutter assembly 20.

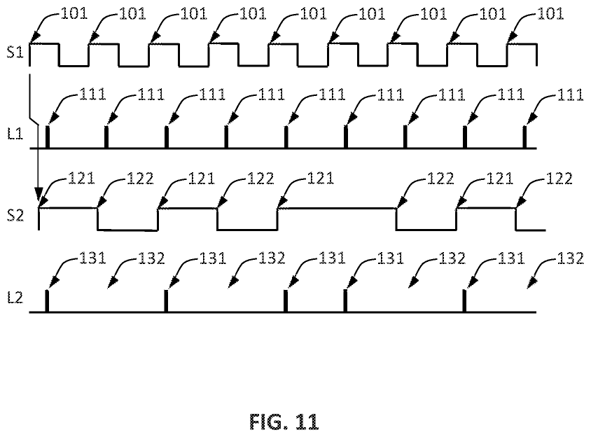

FIG. 11 illustrates an example shutter control process that may be used with laser systems as disclosed herein, such as laser system 10, laser system 11, and laser system 13. Based on the operation of the laser 14, signals as indicated at S1 in FIG. 11 are sent to the trigger input 62 of the controller 60 in advance of each laser pulse emitted by the laser 14. For example, if the laser 14 is being operated at 1 KHz (1000 laser pulses per second), a laser pulse trigger signal 101 is sent to the trigger input 62 of the controller 60 in advance of each laser pulse, at a frequency of 1 KHz. As another example, if the laser 14 is being operated at 900 Hz (900 laser pulses per second), a laser pulse trigger signal 101 is sent to the trigger input 62 of the controller 60 in advance of each laser pulse, at a frequency of 900 Hz. Many other variations are possible. The laser may emit pulses at any frequency suitable for a particular application and may switch frequencies in operation.

The control data processor 64 receives signals from the trigger input 62 indicating the timing of the laser pulses based on the laser pulse trigger signals 101. Based on the signals from the trigger input 62 and the input from the system control 18 regarding the desired output for the laser system, the control data processor 64 sends signals to the shutter motor control 66 to in turn send signals to the shutter motor driver 26 to control the movement of the shutter motor 24 and shutter 22. An example of signals sent from the shutter motor control 66 to the shutter motor driver 26 is indicated at S2 in FIG. 11. The signals 121 cause the shutter motor 24 to move the shutter 22 to a position in which the laser energy is permitted to continue to the output of the laser system toward the target 80, e.g., the first position of the shutter 22 as shown in FIG. 1, 5, 7, or 9. The signals 122 cause the shutter motor 24 to move the shutter 22 to a position in which the shutter position prevents the laser energy from continuing to the output of the laser system toward the target 80, e.g., the second position of the shutter 22 as shown in FIG. 2, 6, 8, or 10.

The timing of the laser pulses emitted by the laser 14 is indicated at L1 in FIG. 11. As shown at L1, a laser pulse 111 is emitted by the laser 14 shortly after each laser pulse trigger signal 101 is sent to the trigger input 62 of the controller 60. The position of the shutter 22 will determine whether or not each laser pulse 111 reaches the output of the laser system.

The laser pulses 131 that reach the output of the laser system are indicated at L2 in FIG. 11. When the shutter 22 has been positioned to permit laser energy to be directed to the laser system output, i.e., after a signal 121 but before a signal 122, the laser pulses are permitted to exit the laser system, indicated by the laser pulses 131. When the shutter 22 has been positioned to disallow laser energy from being directed to the laser system output, i.e., after a signal 122 but before a signal 121, the laser pulses are not permitted to exit the laser system, as indicated by spaces 132 where laser pulses 111 that are present in L1 are not present in L2.

Because the shutter motor 24 can rapidly move the shutter 22 between the first position and second position, the laser system may selectively allow pulses from the laser to reach the system output on a pulse-by-pulse basis. The laser system also may allow groups of laser pulses at a time to reach the system output and may prevent groups of laser pulses at a time from reaching the system output.

In some embodiments, a laser system as described herein may be used for cataract surgery. In some embodiments, the output energy of the laser system may be used for fragmentation or phacoemulsification a cataractous lens. In some examples, the laser output may be used for initial fragmentation of the cataractous lens, followed by phacoemulsification of the lens using an ultrasonic handpiece to complete the breakdown of the lens for removal. In other examples, the laser output may be used for fragmentation or phacoemulsification of the lens to a sufficient degree for removal of the lens without the need for a separate application of ultrasonic energy. Additionally or alternatively, the laser output may be suitable for making corneal incisions and/or for opening the lens capsule.

In other embodiments, the laser system may be suitable for vitreoretinal surgery. In some embodiments, the output energy of the laser system may be suitable for severing or breaking vitreous fibers for removal. In other vitreoretinal applications, the laser output may be suitable for ophthalmic tissue treatment, such as photocoagulation of retinal tissue to treat issues such as retinal tears and/or the effects of diabetic retinopathy.

In one example, the laser operates in the infrared range. For example, the laser may output electromagnetic radiation in the mid-infrared range, for example in a wavelength range of about 2.0 microns to about 4.0 microns. Some examples wavelengths include about 2.5 microns to 3.5 microns, such as about 2.7 microns, about 2.75 microns, about 2.8 microns, or about 3.0 microns. Such a laser may be suitable, for example, for lens fragmentation in cataract surgery, or for other procedures. In another example, the laser emits electromagnetic radiation in the ultraviolet range. In another example, the laser emits electromagnetic radiation in the visible range.

The ability to selectively output laser pulses and/or to control the laser output energy is useful for procedures in which laser control is advantageous. For example, in cataract surgery, it may be desirable to operate the laser system with high power for initially breaking up the lens but with lower power for breaking up smaller pieces. Pulse number control and/or pulse energy level control of laser pulses allows for a correct level of force to be applied to smaller particles which might otherwise be pushed away before they can be aspirated out of the eye by the irrigation system of the hand piece.

As would be understood by persons of ordinary skill in the art, systems and methods as disclosed herein have advantages over prior systems and methods. For example, in some prior systems and methods, selecting pulses can require a large amount of power and can result in an undesired loss of laser power. Pockels cells systems use crystals that rotate the polarity of the laser beam by applying high voltage to the crystals. The high voltage can vary from 0 to 6.5 KV based on the amount of polarity rotation needed. By contrast, systems and methods as described herein can select laser pulses with low power use and with no or essentially no undesired loss of laser power. Furthermore, the cost of systems as described herein may be substantially less than certain other systems. Also, in some embodiments, no high voltage is needed, which improves electromagnetic compatibility for the system.

Persons of ordinary skill in the art will appreciate that the embodiments encompassed by the disclosure are not limited to the particular example embodiments described above. While illustrative embodiments have been shown and described, a wide range of modification, change, and substitution is contemplated in the foregoing disclosure. It is understood that such variations may be made to the foregoing without departing from the scope of the disclosure. Accordingly, it is appropriate that the appended claims be construed broadly and in a manner consistent with the disclosure.

What is claimed is:

1. A laser system comprising:
a laser configured to emit electromagnetic radiation; and
a laser shutter assembly comprising a shutter and a shutter motor;
wherein the shutter motor is configured to move the shutter in an alternating manner between a first position in which electromagnetic radiation emitted by the laser is allowed to be output from the laser system and a second position in which electromagnetic radiation emitted by the laser is prevented from being output from the laser system.

2. The laser system as recited in claim 1, wherein in the first position the shutter is positioned out of a path of electromagnetic radiation emitted by the laser and in the second position the shutter is positioned in the path of the electromagnetic radiation emitted by the laser.

3. The laser system as recited in claim 1, wherein in the first position the shutter is positioned in a path of the electromagnetic radiation emitted by the laser and in the second position the shutter is positioned out of the path of electromagnetic radiation emitted by the laser.

4. The laser system as recited in claim 1, wherein in the first position the shutter is positioned in a first orientation in a path of the electromagnetic radiation emitted by the laser and in the second position the shutter is positioned in a second orientation in the path of electromagnetic radiation emitted by the laser, wherein the second orientation is different from the first orientation.

5. The laser system as recited in claim 1, wherein the laser is configured to emit electromagnetic radiation in pulses.

6. The laser system as recited in claim 5, further comprising a controller adapted to send signals to a shutter motor driver to control moving the shutter between the first position and the second position.

7. The laser system as recited in claim 5, wherein the shutter comprises a mirror.

8. The laser system as recited in claim 7, wherein the shutter motor comprises a galvanometer motor.

9. The laser system as recited in claim 8, wherein the galvanometer motor is configured to move the mirror between the first position and the second position by rotating the mirror about a mirror axis by a selected angle.

10. The laser system as recited in claim 9, wherein the mirror axis and the path of the electromagnetic radiation adjacent the mirror are in a skew line relationship with respect to each other.

11. The laser system as recited in claim 5, further comprising a laser energy control system configured to regulate the amount of electromagnetic energy of each laser pulse that exits the laser system.

12. The laser system as recited in claim 11, wherein the laser energy control system comprises:
a waveplate;
a waveplate motor; and
a polarizer plate;
wherein the waveplate motor is configured to move the waveplate into different positions corresponding to different percentages of laser electromagnetic energy permitted to pass through the laser energy control system.

13. A method of controlling a laser system comprising:
emitting electromagnetic radiation from a laser in a path; and
moving a shutter in an alternating manner between a first position in which electromagnetic radiation emitted by the laser is output from the laser system and a second position in which electromagnetic radiation emitted by the laser is not output from the laser system.

14. The method of controlling a laser system as recited in claim 13, wherein the step of emitting electromagnetic radiation from the laser in a path comprises emitting electromagnetic radiation from the laser in pulses.

15. The method of controlling a laser system as recited in claim 14, further comprising sending signals to a shutter motor driver from a controller to control moving the shutter between the first position and the second position.

16. The method of controlling a laser system as recited in claim 14, wherein the shutter comprises a mirror.

17. The method of controlling a laser system as recited in claim 16, wherein the shutter motor comprises a galvanometer motor.

18. The method of controlling a laser system as recited in claim 17, wherein the step of moving the shutter in an alternating manner between the first position and the second position comprises the galvanometer motor rotating the mirror about a mirror axis back and forth between the first position to the second position.

19. The method of controlling a laser system as recited in claim 14, further comprising moving a waveplate in the path of the electromagnetic radiation emitted by the laser into different positions to regulate the amount of electromagnetic energy of each laser pulse that exits the laser system.

20. The method of controlling a laser system as recited in claim 19, wherein the different positions of the waveplate correspond to different percentages of laser electromagnetic energy permitted to be output from laser system.
